(19) United States
(12) Patent Application Publication
(10) Pub. No.: US 2004/0062809 A1
(43) Pub. Date: Apr. 1, 2004

(54) Biocompatible polymer with a three-dimensional structure with communicating cells, a process for its preparation, and application in medicine and in surgery

(75) Inventors: Jiri Honiger, Villejuif (FR); Andre Apoil, Mantes La Jolie (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Universite Pierre et Marie Curie (Paris VI), Paris (FR)

(21) Appl. No.: 10/329,651

(22) Filed: Dec. 26, 2002

Related U.S. Application Data
(63) Continuation of application No. PCT/FR01/02007, filed on Jun. 25, 2001.

(30) Foreign Application Priority Data
Jun. 26, 2000 (FR) 0008186

(51) Int. Cl. A61K 9/14

(52) U.S. Cl. 424/486

(57) ABSTRACT

Disclosed are porous biocompatible polymers in the form of hydrogels, methods of preparing the hydrogels, and methods of using the hydrogels, e.g., in vitro cell culture and body implants.

### FIELD OF THE INVENTION

The present invention relates to the field of biomaterials.

More particularly, it relates to a process for preparing a biocompatible porous polymer with communicating cavities with controlled size, porosity and stiffness. In particular, it relates to the application of these biocompatible materials to in vitro cell culture, and to preparing seeded biocompatible supports as is or encapsulated by a polymer or its semi-permeable hydrogel that is also biocompatible, as implants in different human or animal tissues or organs, to permanently or temporarily replace a failing organ and thus create a bio-artificial organ. It could be a bio-artificial pancreas, bio-artificial liver, bio-artificial cornea, bio-artificial articular cartilage or bio-artificial bone, etc.

It also relates to the application to the production of transfected cell supports producing a tissue or cell growth factor, more generally a biologically active substance such as a cytokine, a growth factor or a recombinant molecule of therapeutic interest.

This porous material with communicating cavities can also be implanted “naked” into the living body to overcome a substance deficit, for example cartilaginous substance in maxillofacial surgery or for the production of mammary prostheses.

The bio-materials of the invention can also be applied to the preparation of filters for bio-purification of biological fluids, or as enzyme supports to produce an enzyme reactor.

### BACKGROUND OF THE INVENTION

Different porous materials based on natural or synthetic, organic or mineral products have been described for their use in in vitro cell culture and in transplanting living cells. Examples are the use and development of two ceramics based on calcium phosphate: hydroxyapatite (HAP) and β-tricalcium phosphate (TCB). Phosphocalcium hydroxyapatite with formula Ca₁₀(PO₄)₆OH₂ is a synthetic material sold by TECHNIMED as a synthetic bone substitute. The difficulty with that type of material is synthesizing a HAP with just the right pore size so that colonization by cells, in particular bone cells, can occur properly. Further, the use of such material types is limited by a lack of knowledge regarding degradation mechanisms, their durability and their fracture resistance, their surface activity and calcification possibilities.

A number of natural or synthetic polymers have also been described. Examples that can be cited are polyester or polytetrafluoroethylene felts used alone and treated with a polyurethane (1); polyethylmethacrylate/tetrahydrofurfurylmethacrylate (2) and (3), collagen sponges (4); polyhydroxyethylmethacrylate (5); and copolymers of polyglycolic and polylactic acids (6).

Polyamides and/or polyaminoacids that can be used as slow release agents for drugs have also been described.

Other porous materials have been designed for medicine or surgery.

An original method for producing a cellulose sponge was provided in 1996 by O. Pajulo et al., (9). The principle of this method was to coagulate the suspension containing crystals and dendrites of Glauber's salt and of the cellulose solution. The pore size and thickness of the wall between the pores depends on the crystal size and their quantity. This “sponge” was then implanted subcutaneously in the rat to study cellular re-growth.

In 1997, Shapiro L and Cohen S (10) prepared a rigid alginate sponge for seeding with cells followed by culture and transplantation into the living body.

The major portion of these materials constitute bioresorbable materials, which disappear over a greater or less period after implantation into a living organism, leaving cells and cellular tissue in place.

The principal problems connected with the use of such polymers do not solely concern biocompatibility at the material/tissue interface. For non-resorbable polymers, instability to gamma radiation or reactivity to certain types of drugs or certain metabolites can be cited. It also appears to be extremely difficult to avoid the constitutional variability of each production batch. Calcification risks, risks linked to additives, to low molecular weight components, and to in vivo degradation products arise.

For bio-resorbable polymers, there is a severe dearth of information regarding degradation and bio-resorption as well as on the biological effects of the degradation products.

The use of organic or mineral, natural or synthetic polymers or copolymers is currently globally still poorly controlled both regarding reproducibility of production, biocompatibility and biological-metabolic effects on the cells or tissues with which they come into contact.

There is still a need for a biocompatible material that is suitable for the growth of eukaryotic cells, and which does not suffer from the disadvantages described above. The production of such a material turns on the use of a material that is already known for its biocompatibility properties and in particular its haemocompatibility properties, to obtain a structure that is suitable for different uses, both for in vitro cell culture and for implantation into a living organism. For said implantation, both as an artificial organ and for regenerating bone or cartilage tissue, a process must be capable of application to the bio-material employed in order to produce a three-dimensional structure containing multiple cavities communicating with each other and with a the surface of the body in the proximity of the cavities, the size and organization of which can be controlled to allow seeding, growth and cell differentiation if appropriate. After colonizing the space constituted by these cavities, the cells can differentiate by the action of growth factors or differentiation factors that are added or produced by the tissues or organs with which they are in contact.

The different applications also necessitate the use of a process for controlling cavity size, form and rigidity (greater or lesser flexibility) of the polymer.

### SUMMARY OF THE INVENTION

In accordance with the invention, these objectives are achieved by dint of a copolymer from a polymer family used for a number of years in the form of membranes for haemodialysis or in its hydrogel form, for ocular implants or for the preparation of an artificial pancreas (7). Said copolymer has already been shown to be biocompatible and haemo-compatible, and in particular regarding its capacities of not activating the complement system (15), of not inducing leukocyte drop and of only inducing minimal hypoxaemia (8). The polymer in question is a copolymer known as AN 69, manufactured by HOSPAL R & D Int (Meysieu, France).

The process of the invention uses the very properties of producing a hydrogel illustrated in the case of a copolymer of acrylonitrile and sodium methallylsulphonate, said production comprising, in succession, a solution step and a step for gelling then forming a hydrogel. The formation and definition of hydrogels has been defined by Honiger et al., (7). A hydrogel is formed by precipitating a homogeneous polymer solution. On a ternary diagram (polymer/solvent/non-solvent), the equilibrium curve separates a zone in which all of the components are miscible from another zone in which two phases are formed (a solid polymer-rich phase and a liquid phase that is low or depleted in polymer). During hydrogel formation, the system changes from the initial solution of polymer to a composition in which all of the solvent has been replaced by the non-solvent, this transforming the gel into a hydrogel; this hydrogel essentially comprises only non-solvent and polymer. This succession of steps (liquid form, gelled form), the change from the liquid form to the gel form being triggered by contact of the copolymer with a non-solvent means that producing such a gel around a matrix with a pre-selected form and porosity that is selected as a function of the subsequent application of the biocompatible copolymer can be envisaged. In other words, the concept at the basis of this invention is to use a mould or a matrix that endows the bio-material with the selected porosity and form, the rigidity being determined by the conditions for producing the hydrogel and in particular its water content.

The process resides in an essential characteristic of the polymer, namely the capacity of changing from a liquid state to a non liquid state, with a certain rigidity. The present invention is applicable by equivalence to any biocompatible polymer that, thanks to a triggering factor, can change from a liquid state to a non liquid state. The term “non liquid state” means a gel or crystalline or pseudo-crystalline state, or a hydrogel.

The choice of mould or matrix used to endow the bio-material with its form and porosity is made using two alternative strategies. The first strategy is to select a material for the mould or matrix with complete neutrality and biocompatibility. As mentioned above, however, no currently known material exhibits all the properties of controlled porosity, biocompatibility and control of long term effects. The second alternative is to use any substance as a mould or matrix the size and porosity of which can be controlled and which can be eliminated after forming the hydrogel or solid structure on said mould. This elimination can be achieved by dissolution, or by enzymatic digestion.

Total elimination of the mould or matrix prior to in vivo use of the bio-material is preferred. Clearly, then, in this case only the hydrogel or stiffened polymer remains. Its form and porosity are pre-determined by the mould or matrix and its rigidity is determined by the water content; the set of biocompatibility properties already described for AN 69 and the hydrogel AN 69 over several years and many publications are retained in the three-dimensional structures obtained.

In a first implementation, the invention provides a process for producing a porous three-dimensional structure with communicating cavities constituted by a biocompatible polymer comprising a liquid state and a gelled or solid state, comprising the following operations:

preparing a frit with a pre-selected geometry and porosity constituted by a hydrosoluble or hydrolyzable substance which is not soluble in the polymer solvent and which is soluble in the polymer non-solvent;

preparing a solution comprising a polymer in the polymer solvent;

impregnating said frit with the polymer solution;

placing the frit impregnated with the polymer solution under physical conditions for transforming the biocompatible polymer from the liquid state into the gelled or solid state, or a hydrogel incorporating said hydrosoluble or hydrolyzable substance;

dissolving or hydrolyzing the frit, as appropriate, by immersing the mixture in a polymer non-solvent;

recovering the polymer with the selected geometry and porosity in the gelled or solid form or in the form of a hydrogel.

In a further implementation, the invention provides a process for producing a porous three-dimensional structure with communicating cavities constituted by a biocompatible polymer comprising a liquid state and a gelled or solid state, comprising the following operations:

preparing a hydrosoluble or hydrolyzable substance which is not soluble in the polymer solvent and which is soluble in the polymer non-solvent, in the form of particles with a selected size and geometry;

preparing a solution comprising the polymer in the polymer solvent;

forming a heterogeneous mixture containing a solution of the polymer and the hydrosoluble or hydrolyzable substance in a mould;

placing the mould containing the heterogeneous mixture under physical conditions for transforming the biocompatible polymer from the liquid state into the gelled or solid state;

unmoulding the gelled or solidified or hydrogel mixture incorporating said hydrosoluble or hydrolyzable substance;

as appropriate, dissolving or hydrolyzing the hydrosoluble or hydrolyzable substance in a polymer non-solvent;

recovering the polymer with the selected geometry and porosity in the gelled or solid form or in the form of a hydrogel.

The mould intended to produce the form and dimensions of the three-dimensional structure can, for example, be formed from a silicone elastomer. The two implementations of the process described above have two essential characteristics:

a) the biocompatible polymer used must have the properties of changing from a liquid state into a gelled or solid state, this transformation possibly being controlled by an external trigger that is a polymer non-solvent, or temperature or pH, for example, i.e., the property of forming a hydrogel;

b) use of a matrix that acts as a mould to endow the biocompatible polymer with the desired form, said matrix or said mould possibly being eliminated either by dissolving or by hydrolysis.

### BRIEF DESCRIPTION OF THE DRAWINGS

FIGS. 1A and B are photographs taken through an optical microscope of basal bone and articular cartilage surface regrowth into porous material with a three-dimensional structure that has previously been seeded with rabbit chondrocytes and implanted on the femoral condyl after creating a cartilage deficit. The magnification in FIG. 1a is 15 times and that in FIG. 1b is 60 times showing the central portion of photograph 1a showing the cartilage regrowth in detail.

FIGS. 2A and B are photographs of a foam of AN 69 polymer obtained by the process of Example 4 below. The magnification in the top photograph (2a) is ×17 and that of the bottom photograph (2b) is ×100.

### DETAILED DESCRIPTION OF THE INVENTION

Throughout the present text, the term “bio-materials” should be understood to mean non living materials used in a medical device intended to interact with biological systems. By definition, a bio-material is suitable for contact with living tissues or fluids or tissues of the living body. Such contact, which is obvious in the case of an implant, must be extended to the contact which occurs on the surface or outside the human or animal body, for example that occurring with blood in haemodialysis or with the cornea in contact lenses. It is also extended to materials used in biotechnology, in particular materials for in vitro culture of animal or plant cells. A bio-material is biocompatible by nature. The term “biocompatibility” means the capacity of a material to be used with a host response that is appropriate to a specific application. This definition implies “negative” properties such as an absence of toxicity, absence of inflammatory reaction, an absence of complement activation, and an absence of leukocyte drop. It also includes “positive” properties which imply that the material is not necessarily the most inert possible but in contrast, causes the living tissue to react and contributes to the metabolic activation of cells in contact with it or tissues into which it is implanted; this is particularly the case with osteo-conductive materials which facilitate bone growth.

The porous bio-materials of the present invention belong to the category of organic polymers or copolymers. The process of the invention and the material with communicating cavities obtained by the process allows cultured cells to be organized into these communicating cavities and to proliferate if appropriate. These properties not only allow the culture of cells and the manufacture of artificial organs, but also allow the construction of transportable and transplantable cell tissues, in particular for transplantation of neo-cartilage tissue, produced from cultured chondrocytes.

The expression “selected form” means the external shape that can be selected as a function of the geometry desired for an implant. By way of example, an implant for bone regeneration must have the geometry desired for a perfect fit at the insertion site. The form can be produced either by initially forming the matrix constituted by the hydrosoluble or hydrolyzable substance into the desired shape, this constituting the first implementation of the process, or by preparing the hydrosoluble or hydrolysable substance in the form of beads or microbeads the size of which is selected as a function of the desired size for the communicating cavities; these beads or microbeads are then mixed with the biocompatible polymer liquid, said mixture being prepared in a mould of the selected geometry and size. After gelling and solidification of the polymer, the mould is then removed before or after dissolving or hydrolyzing the substance.

In a preferred implementation, the gelling or solidification step is carried out by immersion in a bath containing a polymer non-solvent. As will be shown in the examples, depending on the process which is known per se, the gelling or solidifying bath or hydrogel formation bath comprises water or an aqueous solution of a biologically acceptable salt.

The Applicants have discovered that, surprisingly, under certain conditions, certain materials have considerable advantages more particularly in the field of implants. The bio-materials in question fall into the hydrogel category. Hydrogels are three-dimensional hydrophilic networks which are capable of absorbing large quantities of water or biological fluid and which to a certain extent resemble biological tissue. They are insoluble because of the presence of a network of chemical or physical bonds, and can be formed in response to a large number of physiological or physical stimuli such as temperature, ionic strength, pH or contact with solvents.

In a preferred implementation, the three-dimensional structures are essentially based on a hydrogel, i.e., the structure is constituted by a homogeneous material.

In the process of the invention, the polymer solution comprises at least:

a polymer or copolymer that is soluble in inorganic or organic polar aprotic solvents; and

an organic or inorganic polar aprotic solvent for the polymer or copolymer, said solvent preferably being compatible with the non-solvent used, i.e., miscible with the non-solvent, preferably to an extent of 0 to 100%.

The term “aprotic solvent” means any solvent that does not exchange protons with the surrounding medium or substances dissolved therein.

In a preferred implementation of the invention, a preferred hydrogel contains 50% to 98% of water. The ionic strength of the hydrogel can be in the range 0 to about 500 mEg/kg, preferably in the range 30 to 300 mEg/kg, more preferably in the range 100 to 270 mEq/kg of hydrogel. Low ionic strengths (of the order of 0) are achieved for a hydrogel of the homopolymer PAN (AN69 with no sodium methallylsulphonate group). Such hydrogels can be formed from a solution of polymers comprising at least:

a copolymer of acrylonitrile and an unsaturated olefinic co-monomer carrying anionic groups, said co-monomer being selected from the group formed by methallylsulphonic acid, methallylcarboxylic acid, methallylphosphoric acid, methallylphosphonic acid, methallylsulphuric acid, which may be in their salt forms;

an organic or inorganic polar aprotic solvent for the copolymer.

However, it is possible for the solution of the polymer in the solvent to additionally contain a polymer non-solvent.

In a still more preferred implementation of the invention, the copolymer is a copolymer of acrylonitrile and sodium methallylsulphonate. This polymer has been described and used as a biocompatible material in a number of applications. This polymer is AN69 as referred to hereinbefore. It is a poly(acrylonitrile-sodium methallylsuophonate) copolymer with a molecular weight of about 250000. Its anionic nature depends on the sulphonic group content (3.3 mol %). This copolymer can be dissolved in an aprotic solvent such as N,N-dimethylformamide (DMF), dimethylsulphoxide (DMSO), N,N-dimethylacetamide (DMAA) and propylene carbonate (PC). Starting from the polyacrylonitrile-sodium methallylsulphonate, it is possible to form a hydrogel by precipitating a homogeneous solution in a precipitation bath (phase inversion or phase separation) using a process as described by J Honiger et al (7).

In the process of the invention, the polymer can also be selected from the group formed by polysulphone, polyethersulphone, polyhydroxyethylmethacrylate, polyhydroxypropylmethacrylate, or copolymers thereof.

Depending on the application, the hydrogel can contain 2% to 50% of acrylonitrile copolymers and an unsaturated co-monomer carrying anionic groups, the acrylonitrile/co-monomer mole ratio being in the range 90:10 to 100:0. For a suitable solvent and non-solvent for such a copolymer, a solvent/non-solvent ratio in the range 500:1 to 0.5:1 by weight is required. Such a hydrogel has a microporous structure and an ionic strength in the range 0 to 50 mEq per kilo of gel, with a water content in the range 50% to 98%.

This polymer has been used for more than twenty years as a renal dialysis membrane in the form of hollow fibres or flat sheets. Its physical and chemical properties are well known and for more than twenty years it has been providing excellent biocompatibility with blood and with serum. In particular, since 1978, it was established that AN 69 membrane does not cause complement activation giving rise to aggregation of leukocytes nor to sequestration of the aggregates formed in the pulmonary micro-circulation, leading in turn to leukopenia and to a risk of hypoxia (11).

In the process of the invention, the aprotic solvent for the copolymer is preferably, when the polymer is a copolymer of acrylonitrile with a methallylsulphonate co-monomer, selected from the group formed by N,N-dimethylformamide (DMF), dimethylsulphoxide (DMSO), N,N-dimethylacetamide, polypropylene carbonate and N-methylpyrrolidone (NMP). DMSO and DMF are preferred for the familiarity in use.

The respective proportions of each of the elements composing the solution of polymers can vary depending on the expected characteristics for the biocompatible polymers, in particular as regards its rigidity. By way of example, a material in accordance with the invention comprising 5% to 15% of the polymer will produce a flexible, deformable sponge. However, a material containing 25% to 35% of polymer will be preferred and can produce a porous substance with controlled rigidity/flexibility as a function of the weight ratio of the polymer or copolymer and of the hydrosoluble or hydrolyzable substance.

In the process of the invention, a frit with a geometry or porosity prepared with a hydrosoluble or hydrolyzable substance or this same substance prepared in the form of particles with a selected size and geometry is impregnated or mixed with the biocompatible polymer in its liquid state.

Preferably, the processes of the invention are essentially carried out without evaporation of the solvent or non-solvent.

In a preferred implementation of the invention, the hydrosoluble or hydrolyzable substance that is non soluble in the polymer solvent and soluble in the polymer non-solvent is agglomerated or crystalline saccharose.

In a more preferred implementation of the invention, this substance can be an agglomerate of pseudo-crystals of cane sugar or beet in pieces or as a powder. The advantages of using this substance are its perfect tolerability as regards toxicity, its very high solubility and finally, the facility with which its form and the size of the agglomerated particles can be modified.

Regarding the last point, the use of saccharose means that particles can be obtained with a mean diameter in the range 0.1 to 3 mm, endowing the biocompatible polymer with communicating cavities of the desired size. In some cases, after eliminating the saccharose, the cavities may shrink to some extent. This is homogeneous in the foam obtained and reproducible for a given polymer or copolymer. The skilled person can then select the particle size as a function of the desired size of the communicating cavities.

In the process of the invention, the polymer non-solvent is an aqueous solution of an organic or inorganic salt. Preferably, and in the case in which the polymer solution is composed of copolymers of acrylonitrles and sodium methallylsulphonate, the aprotic solvent being DMSO, the non-solvent for said polymer that is capable of forming the hydrogel is a solution of sodium chloride containing 9 g per litre at ambient temperature, about 20° C.

Finally, in the process of the invention, the hydrosoluble or hydrolyzable substance is, if necessary, eliminated by immersion in distilled water at a temperature in the range 30° to 50°, preferably with stirring. The water is renewed until the sugar crystals are completely dissolved, and the communicating cavities are liberated. The mean diameter of the cavities can be in the range 0.1 to 3 mm.

The cavity diameter clearly depends on the size of the particles of hydrosoluble or hydrolyzable substance that are eliminated, and can be smaller because of shrinkage observed during hydrogel formation.

This set of operations can produce a biocompatible polymer with a selected geometry and porosity that can be used in many in vitro, ex vivo and in vivo applications.

The present invention also provides a porous three-dimensional structure with communicating cavities constituted by at least one biocompatible polymer, which can be obtained by a process as described above; said structures comprise multiple cavities, which communicate with each other and with the surface of said structure. Said three-dimensional structure, based on a porous hydrogel with communicating cells, can be qualified as a “foam”. The term “foam” qualifies both the existence of cavities that communicate mutually and with the surface of said foam, and a variety of rigidity and geometrical properties.

Throughout the present text, the term “foam” or “polymer foam” is used to designate any three-dimensional structure that can be obtained by the process of the invention.

Preferably, the polymer foams of the invention are foams of hydrogel and more preferably, foams of AN 69 obtained by the process.

The hydrogel foams of the invention, more particularly foams of AN 69, can comprise functionalized residues that can form covalent bonds with organic residues. By way of example, said functionalized residues can be —CHO, —NH₂, —COOH or —SH residues. One example of such a functionalization has been described for AN 69 in PCT patent application PCT/FR98/00066. This example is not limiting, however, since International patent applications WO-A-92/07023 and WO-A-92/07006, describe functionalizing other uncharged hydrophilic polymers such as polyethylene glycol-hypoxy covalently bonded to a polyethyleneimine.

The advantage of foams of the invention carrying functionalized residues is the possibility of coupling organic ligands via covalent or ionic bonds; by way of example, such ligands can be selected from the group formed by antibodies, antigens, peptides, proteins or glycoproteins, hormones, enzymes, co-factors thereof, substrates or inhibitors thereof, polysaccharides, lectins, toxins or anti-toxins, nucleic acids or polynucleotides, haptenes or haptene ligands, pigments or colorants. It will be clear to the skilled person that this type of functionalized foam onto which ligands of the type cited above can be fixed has the advantage when a substance or a metabolite present in a biological fluid or in an organ is to be purified, separated or transformed.

The present invention pertains to the use of such functionalized foams as modules for in vitro, ex vivo or in vivo affinity biopurification of biological molecules or macromolecules.

The size and geometry of the communicating cavities of the biopolymer foams can be selected as a function of cultured cells and their organization in the communicating cavities, more particularly when said cells differentiate in the foam. The field of cell culture has been expanding for a long time, and many devices and products have been developed with the aim of optimizing conditions vital to cell culture. Examples are Petri dishes, CO₂ ovens, nutrient media and treating receptacles with products of biological, organic or mineral origin, which allow better organization, adherence, proliferation etc. of the cells during their culture.

However, manipulation with cells cultivated during their implantation or transplantation is not easy. Firstly, they must be placed in suspension to provide immunoprotection my micro or macroencapsulation, but the cells are often already organized and adhere to the dish, which necessitates scraping them off, with a risk of ruining them.

The biopolymer foam defined by the present invention allows the cells to be cultured, to be organized in its communicating cells, to proliferate and to construct transportable and transplantable tissue with or without immunoprotection as will be explained in the examples, in particular when transplanting neocartilage tissue produced by cultured chondrocytes.

Because of the highly compatible character of the polymer in the foams of the invention, these will advantageously comprise animal or plant cells in a medium appropriate to their proliferation and/or differentiation.

One of the first applications of the present invention is the use of this type of foam to culture animal or plant cells, if necessary recombinant, for their in vitro culture and the production of biological macromolecules of interest.

The biocompatible polymer foams of the invention, more particularly hydrogel foams, and still more particularly foams of AN 69 find a particularly advantageous application when they contain cells intended for implanting in the human or animal body. The advantage of the structure of foams with communicating cavities is that when they are seeded with stem cells or undifferentiated cells, it is possible to construct cell tissue in this foam by pre-culture in a medium containing appropriate growth and/or differentiation factors.

A further type of foam of the invention carries chondrocytes or chondrogenic stromal cells. Implanting foams carrying chondrocytes allows a bio-artificial cartilage to be produced, or it can replace a bone deficit. To produce these foams carrying chondrocytes, one means is to separate the chondrocytes of a joint cartilage removed from an animal or human articulation, seed the chondrocytes into the foam with communicating cavities, cultivate these chondrocytes seeded in the support immersed in the nutrient medium in an oven at 37° in an atmosphere comprising 5% CO₂, and after culture, transplant the foam carrying the cells that have proliferated into a joint cartilage in an individual. This transplantation can be autologous or heterologous, i.e., the chondrocytes can originate from a donor individual with tissue compatibility with the receiver (allogenic graft), or can be removed from an individual, cultivated and implanted in the form of a foam carrying chondrocytes in the cartilage or bone to be replaced in that same individual (autologous graft).

In the same manner, the foams of the invention can be seeded with stem cells or cells producing a particular cell line. Marrow is composed of haematopoietic cells in close association with cells of non haematopoietic origin and a support termed the medullar microenvironment. In this non haematopoietic compartment are stromal cells which are cellular progenitors having multipotent characteristics for differentiation towards specific connective tissue such as bone or cartilage. The cells of the stroma and the bone marrow, which represent about 3% of the mononuclear cells, can be isolated by incubating the mononuclear cells with monoclonal antibodies directed against endoglin (CD15) coupled to magnetic beads. This antigen is found in a highly homogeneous cell population with capacities of expansion and chondrogenic properties. The cell suspension can then be isolated using any means that is known to the skilled person, an example of which can be an affinity column attached to a magnet to retain the positive cells which are collected, analyzed and cultured for expansion. This culturing in the foams of the invention is carried out in the presence of a culture medium in the presence of suitable differentiation factors, in particular TGFβ3. Culture under these conditions produces cellular pseudo-tissue that can be implanted into bone or cartilage.

In the same manner, the present invention pertains to a foam of biopolymers with communicating cavities carrying hepatocytes. These foams can then be implanted, for example into the peritoneal cavity. Transplantation of syngenic or congenic hepatocytes allows long term correction of metabolic deficiencies without incurring immunosuppression. An example of the therapeutic potential of transplanting hepatocytes is given by N. Gomez et al. (12). Transplanting biocompatible polymer foam carrying hepatocytes, and more particularly an AN 69 hydrogel, can increase the longevity and tolerability of the transplant.

For this application, it is advantageous in the case of syngenic transplantation to protect the cells with an immunoprotective film or semi-permeable membrane. Such a film or such a membrane may advantageously be a polymer or copolymer hydrogel in accordance with the invention.

In the same manner, the invention also encompasses foams of polymers of the invention carrying islets of Langerhans. The islets of Langerhans can be obtained using any technique that is available to the skilled person at that moment in time. An example that can be cited is the technique described by C Delauney et al. (13). The transplant bearing the islets of Langerhans can be assimilated into a bio-artificial pancreas which, after implanting, produces insulin over a long period and regulates glycemia.

In a further implementation of the invention, the biocompatible polymer foams, and more particularly hydrogel 69, can constitute cell reactors that are implantable in vivo for the production of substances of therapeutic interest. The implant carrying the producing cells can be implanted either sub-cutaneously or into a particular organ or tissue. As an example, it is possible to treat different chronic diseases with therapeutic proteins, for example anaemia with erythropoietin, haemophilia with factor VIII or factor IX, vascular deficiencies with angiogenic factors, or solid tumours with anti-angiogenic factors. The feasibility of such implantation techniques has already been demonstrated by E Payen et al for erythropoietin (14). A mini-bio-reactor in accordance with the invention can also contain cells producing vectors, viruses or recombinant plasmids for gene therapy.

The bio-reactor can be implanted in situ close to the cells that are to be treated by that method. By way of example, implantation into muscle tissue or cerebral implantation can be cited, for the respective treatment of certain genetic disorders or certain cancers.

In a further aspect, the invention concerns the use of foams of biocompatible polymers in accordance with the invention for the production of a prosthesis intended to overcome a substance deficit in an organ, in particular a mammary prosthesis or the complement of bone tissue. In this use, the implanted polymer foam comprises no cells, but a medium that allows cells in contact with said foam to colonize it in situ. After implantation, the cells of the organ into which the foam carrying a suitable sterile culture medium is implanted, can proliferate and assist in overcoming the deficit in the organ.

In a further aspect, the invention concerns the use of foams of biocompatible polymers in accordance with the invention in producing drugs for controlled release of an active principle. Said hydrogel foams offer a particularly suitable means for administering molecular or macromolecular active principles and in particular active principles of a peptide or polypeptide nature.

In general, the three-dimensional foams with communicating cells of the invention are applicable every time the skilled person wishes to produce an implant with undifferentiated or differentiated cells of a certain type. The above examples are not limiting; it is also possible to envisage an implant carrying neuronal cells, keratinocytes, etc.

These foams are also applicable every time that in situ differentiation of stem cells is required prior to implantation, constituting the template for a preformed tissue which could then advantageously be grafted into an organ or tissue the function of which is to be restored.

Finally, they are applicable every time that the use of cell cultures retaining all of their metabolic functions is required; non limiting examples are biosensors intended to evaluate the effect of certain molecules or effectors on cells.

All other applications of this material of the invention are based on the flow of liquids and gas through this porous material with communicating cavities:

1) non laminar flow of liquids in the communicating cavities of this material eliminates the limiting layer in which transfer is achieved by diffusion and as a result, increases direct contact between the liquid and the polymer or its hydrogel, which constitutes the matrix of said material. This can therefore be exploited for liquid purification, for ion exchange in liquids, and for resorption of active substances previously deposited on the cavity surface by liquids or gases that are passed. Clearly, the active substances deposited on the cavity surface can react simply by contact with the flowing fluids or gas and thus stimulate, inhibit or accelerate reactions occurring in the fluids and gas (for example inhibit blood coagulation, catalyse gas synthesis, etc);

2) a further application in this field is the possibility of producing a perfect mixture of a plurality of liquids or gases during their passage through the material.

The examples and photographs below constitute non-limiting illustrations of both the process for producing said three-dimensional biocompatible polymer structures and their in vitro or in vivo use.

### EXAMPLES

### EXAMPLE 1

### Production of a Flexible Foam with Communicating Cavities with Approximate Dimensions of 0.8 Cm×1.3 Cm×2.1 Cm and with a Pore Size of a Few Tens of a Millimetre

A polymer solution constituted as follows was prepared:

6% of acrylontrile and sodium methallylsulphonate copolymer;

3% of an aqueous 9 g/l sodium chloride solution;

91% of dimethylsulphoxide (DMSO);

by successively dissolving the components with stirring and at 50° C. After cooling to ambient temperature, a 50 ml polymethylpentene beaker (TPX) was filled. A 1.2 cm×1.8 cm×2.8 cm piece of cane sugar was taken and slowly and completely immersed in the polymer solution using tweezers. Once all the air trapped in the interstices between the sugar crystals had been released, the piece of sugar was removed from the polymer solution, the excess solution and the entire surface of the sugar was drained and it was immersed for a few minutes in a gelling bath composed of an aqueous 9 g/l solution of sodium chloride at ambient temperature (20° C.). It was then placed in hot (40° C.) distilled water, stirred and frequently replaced so that the sugar crystals dissolved and released from the communicating cavities.

### EXAMPLE 2

### Production of a Semi-Rigid Foam with Communicating Cavities Carrying Chondrocytes

A silicone elastomer mould was prepared comprising a smooth flat bottom surrounded by a 1.5 mm thick bead.

A 25% polymer solution was prepared by dissolving a copolymer of acrylonitrile and sodium methallylsulphonate in DMF. This solution was then mixed in a proportion of 1:4 with sieved cane sugar crystals with a size of 1-1.5 mm. This mixture was spread onto the silicone mould using a spatula to the thickness dictated by the height of the bead. It was then completely immersed in a coagulating bath containing physiological serum. After a few minutes, the plate formed was unmoulded and the sugar crystals were dissolved by washing with distilled water at 40° C. as described in Example 1.

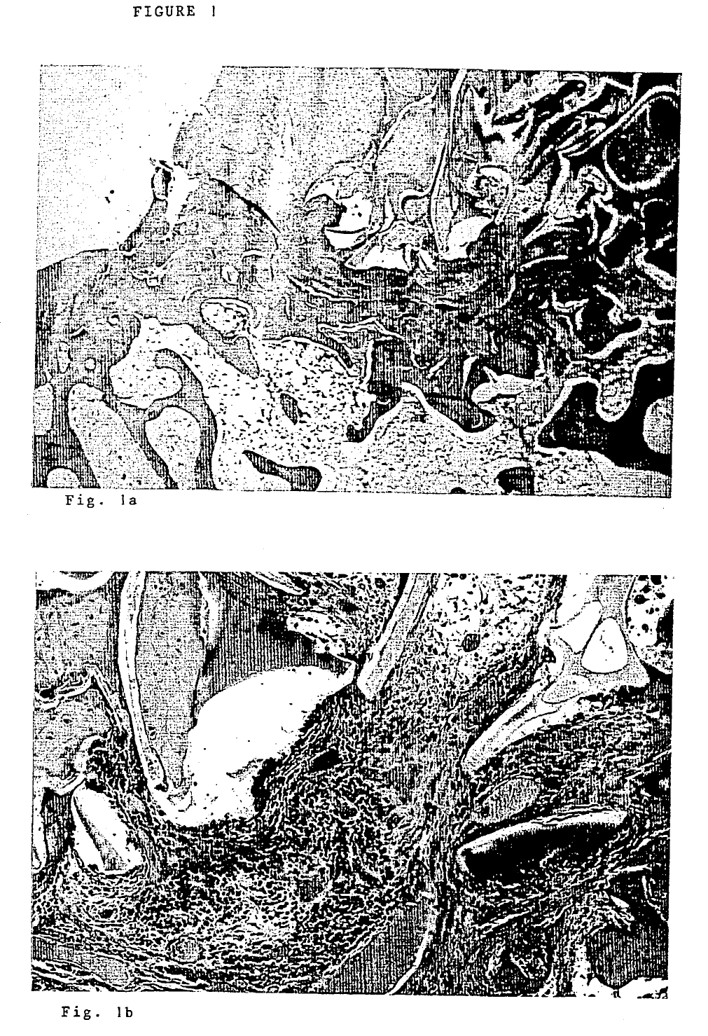

The porous elastomer plate was then decontaminated using a solution containing peracetic acid (APA), and carefully washed with sterile physiological serum until the last traces of APA had disappeared. It was then seeded with chondrocytes isolated from rabbit articular cartilage either by injection using a syringe with a needle or by squeezing and releasing, rather like a sponge. The porous elastomer plate with communicating cells seeded with chondrocytes was placed in a Petri dish containing nutrient liquid and underwent cell culture in a CO₂ oven. Two weeks later, the chondrocytes had organized, proliferated and created a continuous pseudo tissue. The results obtained are shown in the photograph in FIG. 1.

### EXAMPLE 3

### Production of a Thin Porous Material (0.5 Mm) as a Support for Living Cells, in Particular Keratocytes

A homogeneous 25% mixture of the solution of AN 69 polymer in dimethylacetamide (DMAA) and six parts by weight of 0.1-0.5 mm cane sugar was compression moulded between two flat glass plates or were flattened using a glass cylinder, followed by immersing the mould/mixture ensemble in a gelling bath and dissolving the sugar crystals using the method described in Example 1.

### EXAMPLE 4

### Production of a Filter Block from a Polymer with Communicating Cells for Use in a Variety of Treatments for Liquids Circulating in the Pores through the Filter Block

A solution containing 25% of acrylonitrile-sodium methallylsulphonate copolymer and 75% of dimethylformamide was prepared. 9% of this solution was mixed, using a spatula, with 91% of crystalline cane sugar in a glass beaker or a dimethylformamide-resistant plastic beaker. This mixture was then transferred to another glass or dimethylformamide-resistant beaker and the mixture was packed using a curved spatula and/or using a cylinder or another beaker with a slightly smaller diameter which could act as a packing piston. The beaker with the packed mixture was immersed in distilled water or in an aqueous solution composed of a variety of mineral or organic salts, preferably biologically acceptable salts. After a few minutes, the filter block was unmoulded and the sugar crystals were dissolved by continuous or batchwise washing with distilled water or with aqueous solutions of various salts.

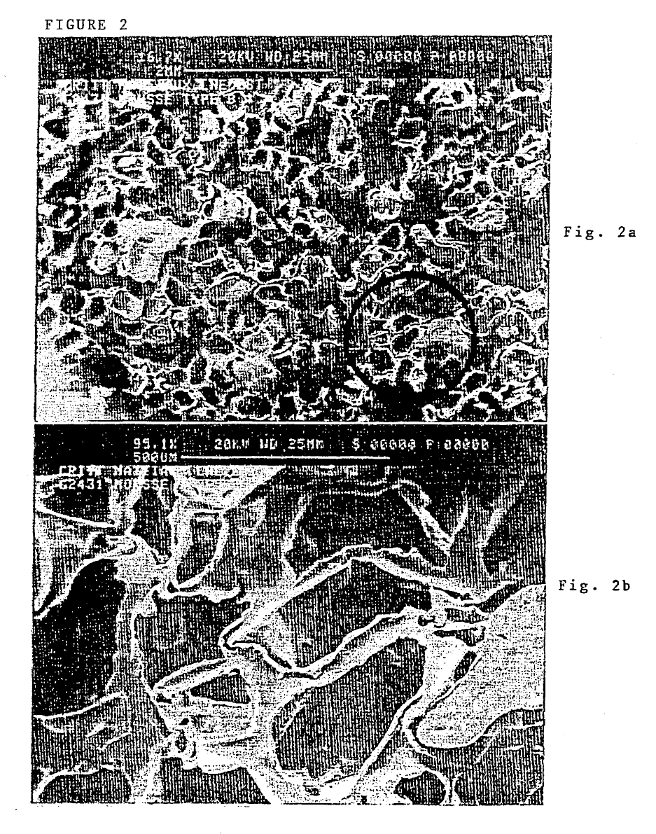

A porous material formed from AN 69 hydrogel with communicating cavities was obtained, containing 78% (by weight) of water in the cavities and with a water content in the hydrogel of about 75% (by weight) (FIG. 2).

### EXAMPLE 5

### Production of a Polymer Form with Communicating Cells by Moulding a Mixture of a Polymeric Solution and Crystalline Sugar

A mixture that was identical to that described in Example 4 was prepared. This mixture was introduced into a glass tube using a spatula and packed using a polytetrafluoroethylene rod. Distilled water was then introduced into the tube and allowed to leave under gravity and a cylinder was released under gravity or a slow flow of water, which cylinder, after completely eliminating the crystalline sugar, became a cylinder of a polymer with communicating cells.

### EXAMPLE 6

### Production of an AN 69 Polymer Block Containing up to 97% Water in the Communicating Cells and Which after the Necessary Examination Can Act as an Implant for Replacing Missing Matter (Mammary Implant, for Example)

A mixture containing 3.5% of the polymeric solution (25% copolymer of acrylonitrile and sodium methallylsulphonate and 75% dimethylformamide), and 96.5% of crystalline sugar was prepared. This mixture was transferred into a mould, packed, then the filled mould was immersed in water or aqueous solutions of various salts. After completely dissolving the sugar, a block of porous elastomer was obtained containing almost 97% water in its cavities.

### EXAMPLE 7

### Production of a Porous Polyethersulphone Material with Communicating Cavities

A homogeneous mixture of a 25% polyethersulphone solution in DMF and ten parts by weight of cane sugar in 0.5-1 mm crystals was moulded, coagulated and the sugar was dissolved exactly as described in Example 4.

### EXAMPLE 8

### Production of a Porous Polyhydroxypropyl Methacrylate Material with Communicating Cavities

A homogeneous 60% mixture of polyhydroxypropyl methacrylate in DMF and ten parts by weight of cane sugar in 0.5-1 mm crystals was moulded, coagulated and the sugar was dissolved exactly as described in Example 4. A flexible polyhydroxypropyl methacrylate material was obtained which had good plasticity and was porous with communicating cavities.

### EXAMPLE 9

### Production of a Flexible Porous Polysulphone Material with Spaced Out Communicating Cavities

A homogeneous 20% mixture of polysulphone in DMF and ten parts by weight of cane sugar in 0.5-1 mm crystals was moulded, coagulated and the sugar was dissolved exactly as described in Example 4.

### EXAMPLE 10

### Production of a Rigid Porous Polysulphone Material with Tightly Packed Communicating Cavities

A homogeneous 20% mixture of polysulphone in DMF and five parts by weight of cane sugar in 0.5-1 mm crystals was moulded, coagulated and the sugar was dissolved exactly as described in Example 4.

It can be seen that by modifying the ratio between the polysulphone/DMF and the cane sugar, 1/10 in Example 9 and 1/5 in Example 10, foams with radically different qualities are obtained, namely flexible with large cavities in the first case, and rigid with narrow cavities in the second case.

### REFERENCES

(1) Messner K., Lohmander L.-S., Gillquist J., “Neocartilage after artificial cartilage repair in the rabbit: histology and proteoglycan fragments in joint fluid”, J. Biomed. Mat. Res. (1993) 21: 949-954.

(2) Reissis N., Kayser M., Bentley G., Downes S., “A hydrophilic polymer system enhanced articular cartilage regeneration in vivo”, J. Mater. Sci.: Mat. in Medicine (1995) 6: 768-772.

(3) Sawtell R., Downes 5., Kayser M., “An in vitro investigation of the PEMA/THFMA system using chondrocyte culture”, J. Mater. Sci.: Mat. in Medicine (1995) 6: 676-679.

(4) Toshia Fujisato, Toshinobu Sajiki, Quiang Liu, Yoshito Ikada, “Effect of basic fibroblast growth factor on cartilage regeneration in chondrocyte-seeded collagen sponge scaffold”, Biomaterials (1996) 17: 155-162.

(5) Reginato A., Iozzo R., Jimenez S., “Formation of nodular structures” resembling mature articular cartilage in long-term primary cultures of human fetal epiphyseal chondrocytes on a hydrogel substrate”, Arthritis & Rheumatism (1994) 37: 1338-1349.

(6) Sittinger M., Reitzel D., Dauner M., Hierlemann H., Hammer C., Kastenbauer E., Planck H., Burmester G., Bujia J., “Resorbable polyesters in cartilage engineering: Affinity and biocompatibility of polymer fiber structures to chondrocytes” J. Biomed. Mat. Res. (1996) 33: 57-63.

(7) Honiger J., Darquy S., Reach G., Muscat E., Thomas M., Collier C., “Preliminary report on cell encapsulation in a hydrogel made of biocompatible material, AN 69, for the development of a bioartificial pancreas”, The International Journal of Artificial Organs (1994), vol. 17,1: 046-052.

(8) Honiger J., Balladur P., Mariani P., Calmus Y., Vaubourdolle M., Delelo R., Capeau J. and Nordlinger B., “Permeability and biocompatibility of a new hydrogel used for encapsulation of hepatocytes”, Biomaterials (1995), 16: 753-759.

(9) Pajulo 0., Viljanto J., Loenberg B., Hurme T., Loenquist K., Saukko P., “Viscose cellulose sponge as an implantable matrix: Changes in the structure increase the production of granulation tissue”, J. Biomed Mat. Res. (1996) 32: 439-446.

(10) Shapiro L., Cohen S., “Novel alginate sponges for cell culture and transplantation”, Biomaterials (1997) 18: 583-590.

(11) Jacob A. I., Gavellas G., Iarco R., Perez G., Bourgoignie J.-J., leukopenia, hypoxia and complement functions with a different hemodialysis membranes kidney Int. (1980) 18: 505-509.

(12) Gomez N., Balladur P., Calmus Y., Baudrimont M., Honiger J., Delelo R., Myara A., Crema E., Trivin F., Capeau J. and Nordlinger B., “Evidence for survival and metabolic activity of encapsulated xenogeneic hepatocytes transplanted without immunosuppression in gunn rats”, Transplantation (Jun. 27, 1997), vol. 63,12: 1718-1723.

(13) Delaunay C., Honiger J., Darquy S., Capron F., Reach G., “Bioartificial pancreas containing porcine islets of langerhans implanted in low-dose streptozotocin-induced diabetic mice: effect of encapsulation medium”, Diabetes & Metabolism (1997) 23: 219-227.

(14) Payen E. Dalle, B., Honiger J., Henri A., Kuzniak L., Rouyer-Fessard P., Benzard Y., “Régulation de la production d'erythropoïetine transgénique in vivo”, European Hemotologic Applications (E.H.A.) Meeting, Barcelona (June 1999).

(15) Honiger J., Couturier C., Goldschmidt P., Maillet F., Kazatchkine M.-D., Laroche L., “A new anionic hydrogel for corneal surgery”, J. Biomed. Mat. Res. (1997) 37: 548-553.

1. A process for producing a porous three-dimensional structure with communicating cavities constituted by at least one biocompatible polymer comprising a liquid state and a gelled or solid state, the biocompatible polymer being a hydrogel, comprising the following operations:
preparing a frit with a pre-selected geometry and porosity constituted by a hydrosoluble or hydrolyzable substance which is not soluble in the polymer solvent and which is soluble in the polymer non-solvent;
preparing a solution comprising a polymer in the polymer solvent;
impregnating said frit with the polymer solution;
placing the frit impregnated with the polymer solution under physical conditions for transforming the biocompatible polymer from the liquid state into the gelled or solid state, or a hydrogel incorporating said hydrosoluble or hydrolyzable substance;
dissolving or hydrolyzing the frit, as appropriate, by immersing the mixture into a polymer non-solvent;
recovering the polymer with the selected geometry and porosity in the form of a hydrogel.

2. A process for producing a porous three-dimensional structure with communicating cavities constituted by at least one biocompatible polymer comprising a liquid state and a gelled or solid state, the biocompatible polymer being a hydrogel, comprising the following operations:
preparing a hydrosoluble or hydrolyzable substance which is not soluble in the polymer solvent and which is soluble in the polymer non-solvent, in the form of particles with a selected size and geometry;
preparing a solution comprising the polymer in the polymer solvent;
forming a heterogeneous mixture containing a solution of the polymer and a hydrosoluble or hydrolyzable substance in a mould;
placing the mould containing the heterogeneous mixture under physical conditions for transforming the biocompatible polymer from the liquid state into the gelled or solid state or hydrogel;
unmoulding the gelled or solidified of hydrogel mixture incorporating said hydrosoluble or hydrolyzable substance;
as appropriate, dissolving or hydrolyzing the hydrosoluble or hydrolyzable substance in a polymer non-solvent;
recovering the polymer with the selected geometry and porosity in the gelled or solid form or in the form of a hydrogel.

3. A process according to claim 1 or claim 2, in which the gelling or solidification or hydrogel formation step is carried out by immersion in a bath containing a polymer non-solvent.

4. A process according to claims 1 to 3, in which the hydrosoluble or hydrolyzable substance that is not soluble in the polymer solvent and soluble in the polymer non-solvent is agglomerated or crystalline saccharose.

5. A process according to claim 1, 2 or 3, in which the biocompatible polymer is a hydrogel containing 50% to 98% of water and with an ionic strength in the range 30 to 300 mEq/kg.

6. A process according to claim 5, characterized in that the hydrogel has an ionic strength in the range 100 to 270 mEq/kg.

7. A process according to one of claims 1 to 6, in which the polymer solution comprises at least:
a polymer or copolymer that is soluble in inorganic or organic polar aprotic solvents;
an organic or inorganic polar aprotic solvent for the copolymer.

8. A process according to one of claims 1 to 7, in which the polymer solution additionally comprises a polymer non-solvent.

9. A process according to any one of claims 1 to 5, characterized in that the polymer is soluble in aprotic solvents that are miscible with the non-solvent.

10. A process according to one of claims 7 to 9, in which the polymer solution comprises at least one copolymer of acrylonitrile and an unsaturated olefinic co-monomer carrying anionic groups, said co-monomer being selected from the group formed by methallylsulphonic acid, methallylcarboxylic acid, methallylphosphoric acid, methallylphosphonic acid and methallylsulphuric acid, optionally in their salt forms.

11. A process according to claim 10, in which the copolymer is a copolymer of acrylonitrile and sodium methallylsulphonate, or AN69.

12. A process according to one of claims 7 to 10, in which the polymer is selected from the group formed by polysulphone, polyethersulphone, polyhydroxyethylmethacrylate, polyhydroxypropylmethacrylate, or copolymers thereof.

13. A process according to one of claims 1 to 12, in which the polymer solvent is an aprotic solvent selected from the group formed by N,N-dimethylformamide (DMF), dimethylsulphoxide (DMSO), N,N-dimethylacetamide and N-methylpyrrolidone (NMP).

14. A process according to one of claims 1 to 13, characterized in that the process is carried out essentially without evaporating off the solvent or non-solvent.

15. A porous three-dimensional structure with communicating cells constituted by at least one biocompatible polymer and comprising multiple cavities, which communicate with each other and with the surface of said structure, said polymer comprising a liquid state and a gelled or solid state and being in the form of a hydrogel.

16. A structure according to claim 15, characterized in that the hydrogel is a hydrogel of a copolymer of acrylonitrile and sodium methallylsulphonate, or AN69.

17. A structure according to claim 15 or claim 16, characterized in that the mean diameter of the cavities is in the range 0.1 to 3 mm.

18. A structure according to one of claims 15 to 17, characterized in that it contains chondrocytes or chondrogenic stromal cells in a medium appropriate for their proliferation and/or differentiation.

19. A structure according to one of claims 15 to 17, characterized in that it contains islets of Langerhans in a medium appropriate for their survival.

20. A structure according to one of claims 15 to 17, characterized in that it contains animal cells, recombinant if appropriate, and which produce substances of therapeutic interest, in a culture medium appropriate for their survival and to their secretion.

21. A structure according to one of claims 15 to 17, characterized in that the biocompatible polymer comprises functionalized residues that can form covalent bonds with organic residues, in particular —CHO, —NH₂, —COOH and —SH.

22. Use of a three-dimensional porous structure with communicating cells according to claim 18, for the production of a bio-artificial cartilage or to replenish a bone deficit.

23. Use of a three-dimensional porous structure with communicating cells according to claim 19, for the production of a bio-artificial pancreas.

24. Use of a three-dimensional porous structure with communicating cells according to claim 20, for the production of an implantable reactor for in vivo production of therapeutic substances.

25. Use according to claim 24, in which the substance is a molecule of therapeutic interest.

26. Use according to claim 24, in which the substance is a recombinant virus or vector carrying a gene of interest, for gene therapy.

27. Use of a three-dimensional porous structure with communicating cells according to claim 21, onto which ligands have been grafted, if appropriate by covalent bonds, as a module for ex vivo or in vivo affinity biopurification of biological molecules.

28. Use of a three-dimensional porous structure with communicating cells according to claim 15 or claim 16, in the production of a prosthesis intended to overcome a substance deficit in an organ, in particular a mammary prosthesis or for replacing cartilage tissue.

29. Use of a three-dimensional porous structure with communicating cells according to claim 15, for the production of a form for controlled release of the active principles of drugs.
